(19) United States
(12) Patent Application Publication
(10) Pub. No.: US 2016/0166720 A1
(43) Pub. Date: Jun. 16, 2016

(54) STERILIZER

(71) Applicant: TOMY INTERNATIONAL, INC., Dyersville, IA (US)

(72) Inventor: Joshua Wiesman, Weston, MA (US)

(21) Appl. No.: 14/125,226

(22) PCT Filed: Aug. 12, 2013
(86) PCT No.: PCT/US13/54484

§ 371 Date: Dec. 10, 2013

Related U.S. Application Data
(60) Provisional application No. 61/681,876, filed on Aug. 10, 2012.

(51) Int. Cl. A61L 2/07

(52) U.S. Cl. CPC A61L 2/07

(57) ABSTRACT

A sterilizer including a base and a lid. The base includes a bottom wall and a sidewall. There is an elastomeric covering over at least most of the outside of the sidewall. The lid is sized and shaped to close the base. The lid defines a vent that is adapted to vent the base.

### FIELD

This disclosure relates to a sterilizer.

### BACKGROUND

Some sterilizers for home use are used by adding water and then placing the sterilizer in a microwave oven such that the water boils, creating steam to thereby sterilize the contents. These sterilizers can be used to sterilize parts of baby bottles and the like. Such sterilizers should be designed such that the user can easily place and remove items to be sterilized.

### SUMMARY

Featured in one example of the disclosure is a sterilizer comprising a base comprising a bottom wall and a sidewall, an elastomeric covering over at least most of the outside of the sidewall, and a lid sized and shaped to close the base, the lid defining a vent that is adapted to vent the base. The lid may comprise a handle. The vent may pass through the handle. The bottom wall may have an interior surface that defines a series of spaced raised areas. The raised areas may be arranged generally concentrically about a central axis. The raised areas may be generally arc-shaped. The raised areas may comprise a plurality of generally arc-shaped segments that are spaced from one another. The segments may be aligned along a series of generally circular axes. The raised areas may be arranged along a plurality of radially-spaced generally circular axes. The raised areas may be arranged such that there is a space between the segments that lie along a single generally circular axis, and wherein the spaces between segments that lie along the respective radially-spaced generally circular axes are generally radially aligned. The raised areas may be at least substantially topped by an elastomeric material.

The base may further comprise a plurality of feet. The base may include a projection, and the elastomeric covering over at least most of the outside of the base may extend over this projection. The base may have one or more thin vertical structures extending upwardly and inwardly from an interior wall of the base, wherein each thin vertical structure has a top portion that is located slightly inwardly of the interior wall so as to define an upper gap between the structure and the interior wall. The gap may be constructed and arranged to act as a receptacle for a relatively thin structure that can be placed into the gap so as to hold the structure off of the bottom of the base.

Another example comprises a sterilizer comprising a base comprising a bottom wall and a sidewall, and an elastomeric covering over at least most of the outside of the sidewall, wherein the bottom wall defines a series of raised areas that are at least substantially topped by an elastomeric material, wherein the raised areas are intermittently formed and oriented generally concentrically about a central axis, and a lid sized and shaped to close the open top of the inner basin, the lid defining a vent that is adapted to vent the base, where the lid comprises a handle and the vent passes through the handle. The base may include a projection, and the elastomeric covering over at least most of the outside of the base may extend over the projection of the base. The base may have one or more thin vertical structures extending upwardly and inwardly from an interior wall of the base, wherein each thin vertical structure has a top portion that is located slightly inwardly of the interior wall so as to define an upper gap between the structure and the interior wall. The gap may be constructed and arranged to act as a receptacle for a relatively thin structure that can be placed into the gap so as to hold the structure off of the bottom of the base.

### BRIEF DESCRIPTION OF THE DRAWINGS

FIG. 1 is a perspective view of a sterilizer according to one example of the present disclosure.

FIG. 2 is a front view of the sterilizer illustrated in FIG. 1.

FIG. 3 is a right-side view of the sterilizer illustrated in FIG. 1.

FIG. 4 is a left-side view of the sterilizer illustrated in FIG. 1.

FIG. 5 is a top view of the sterilizer illustrated in FIG. 1.

FIG. 6 is a bottom view of the sterilizer illustrated in FIG. 1.

FIG. 7 is a top view with the lid of the sterilizer removed.

FIG. 8 is a cross section taken along line 8-8 of FIG. 7.

FIG. 9 is a partial view of one detail of FIG. 8.

FIG. 10 is a partial view of another detail of FIG. 8.

### DETAILED DESCRIPTION

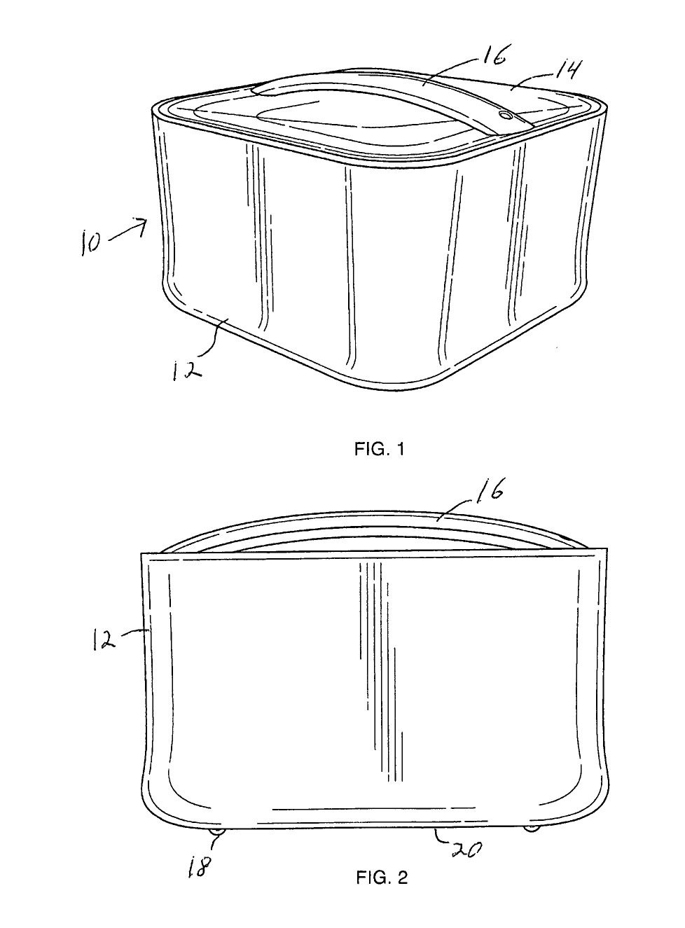

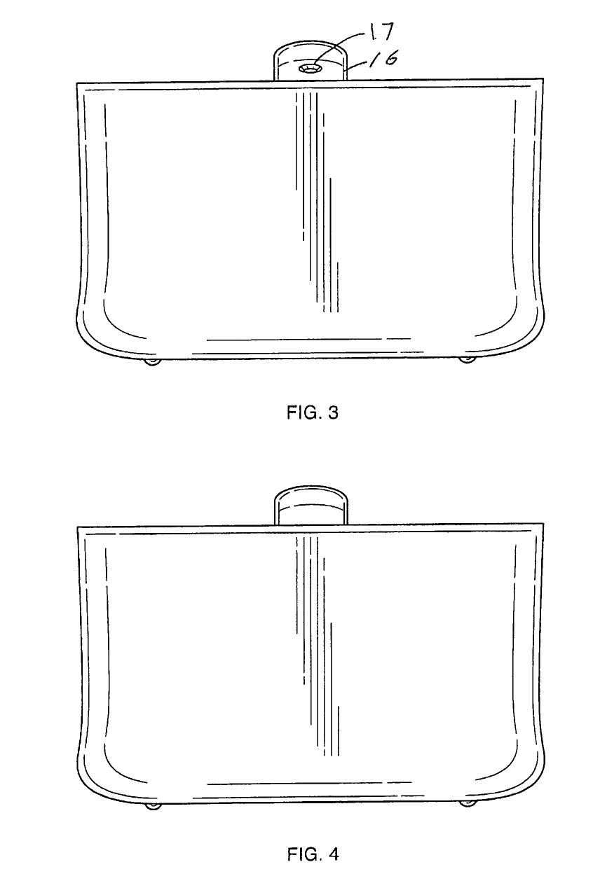

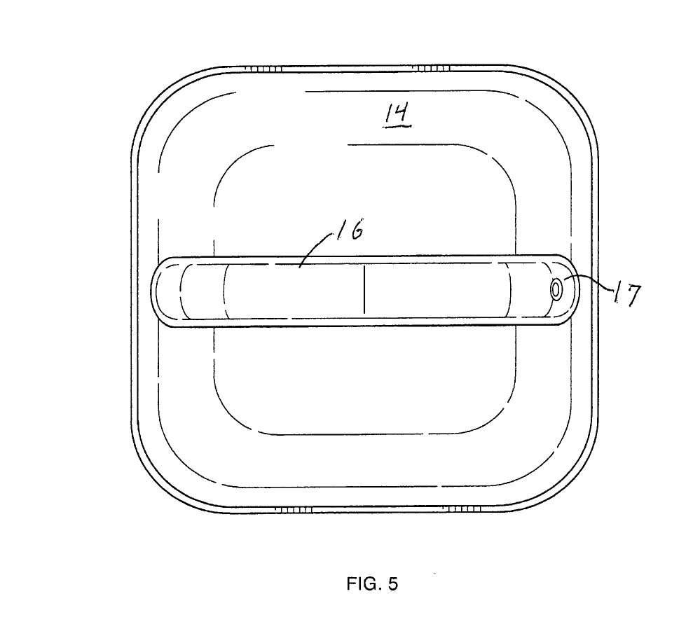

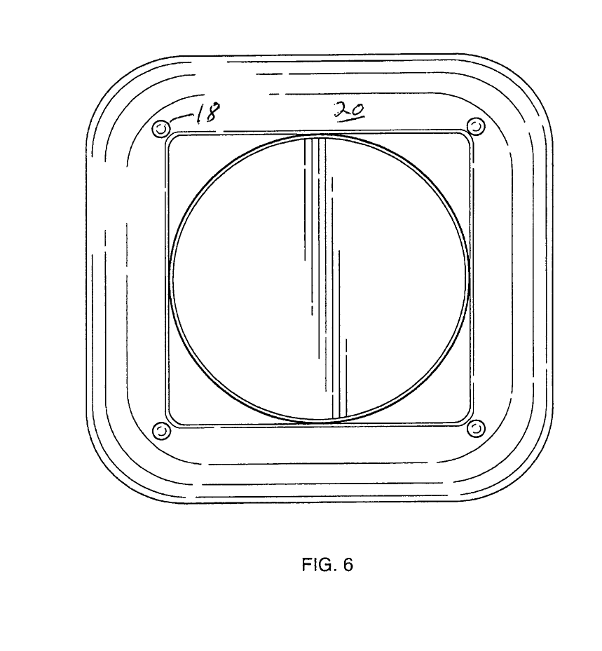

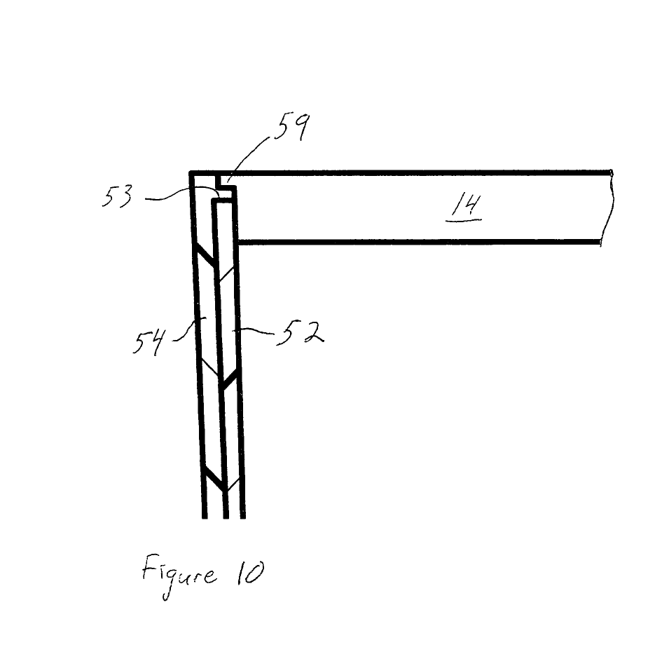

A non-limiting example of a sterilizer is shown in FIGS. 1-10. Sterilizer 10 comprises two separate parts: an open-top base 12, and a removable lid 14 having a handle 16. The base 12 includes a bottom wall 19 and a sidewall 15 that define an interior cavity 21. The sterilizer 10 can be formed using an injection-molding process.

The base 12 includes an interior surface 11 and an exterior surface 13. The base 12 also includes an inner core 52 that defines the interior surface 11 of the base, and an over-molded silicone shell 54 forming the exterior surface 13 of the base, at least along the sidewalls. Since the function of the silicone shell is primarily for purposes of handling/grip and also affects the appearance, the shell does not need to extend over the bottom wall 19 of the base 12. The shell 54 can be colored, to present a desired appearance. The inner core 52 can be made from a plastic material such as polyurethane or polyethylene, or a similar thermoplastic material. The inner core could be made of other plastic materials or materials other than plastic that can withstand sterilization temperatures and conditions; one example is stainless steel. The base 12, in this non-limiting example, has a rounded rectangular cross-sectional shape, and the sidewalls are curved at the bottom and slightly flared toward the bottom as best shown in FIGS. 2-4. Such flared sidewalls are optional, but contribute to the appearance and also offer a better grasping position for the user to place her hands during transportation of the sterilizer 10. However, the base 12 can be made of other suitable materials or have a different shape such as rounded, or more rounded or less rounded than shown in the drawings. The base 12 can include feet 18 that maintain bottom surface 20 raised relative to a countertop or other flat surface on which sterilizer 10 is placed. The feet 18 can be but need not be integrally molded with the base 12.

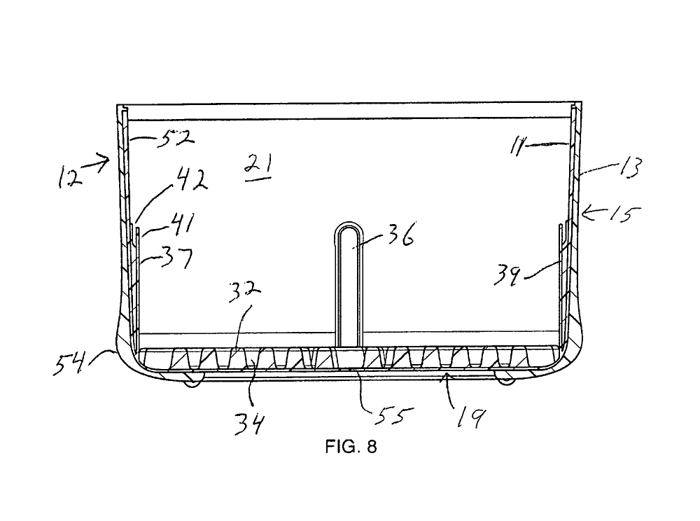

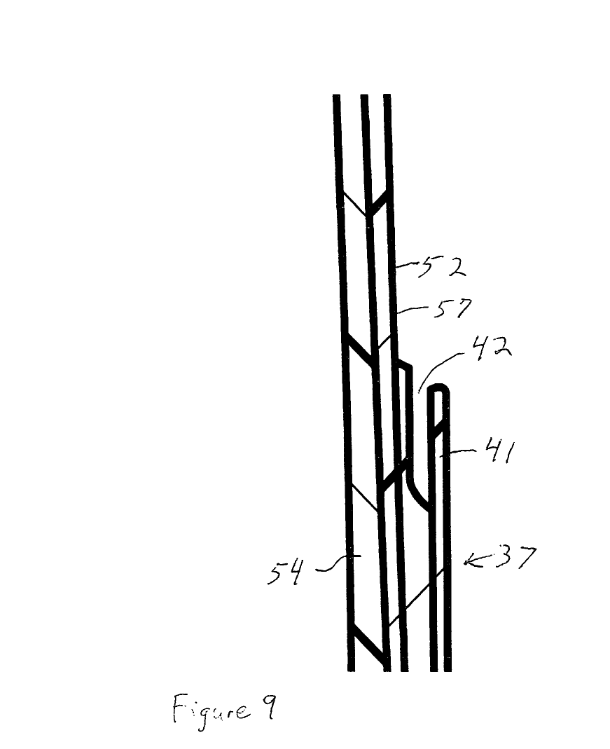

The lid 14 is removably coupled to the base 12 and can be made from the same or similar plastic as is used for the inner core 52. The lid 14 may also include one or more locking features that secure the lid 14 to the base 12. The lid 14 is sized and shaped to fit on a shelf 59 (see FIGS. 8 and 10). The shelf 59 is formed in the base 12 as an inwardly-directed radial projection from an upper portion of the shell 54. The projection or shelf 59 can extend around the periphery of the base 12 or be intermittently formed in particular locations around the base 12. The projection 59 is positioned above a top 53 of the inner core 52. The silicone material of the over-molded silicone shell 54 forms the projection 59. This construction allows the lid 14 to seal onto the silicone projection 59. As silicone is a relatively soft elastomer with grip properties, this arrangement helps to hold the lid 14 in place and also may create a better seal to the steam generated by the sterilizer 10 as opposed to harder mating surfaces. The lid 14 includes a vent 17 (e.g., a through-hole) that is formed in an outer portion of the handle 16, away from where the user would grasp the handle 16. The vent 17 extends entirely through the handle 16 to vent the interior of the sterilizer 10.

The inside of the bottom wall 55 of the inner core 52 of the base 12 comprises a plurality of raised areas 32, and valleys 34 between the raised areas 32. The raised areas 32 and valleys 34 present rounded formations as opposed to more angular corners or edges. This arrangement makes the bottom of the base 12 and the intersections of the raised areas and the bottom easier to clean. The raised areas 32 are preferably topped by or fully covered by, or at least substantially covered on at least on their top surfaces, with a silicone material or another elastomer. The silicone is a thermoplastic elastomer, which provides grip properties that allow the user to grasp the sterilizer 10 with less chance of slippage, and also the silicone on the raised areas 32 presents a grip surface that helps to hold items placed onto the raised areas 32. The valleys 34 provide a volume that accepts water that sits below items that are placed on the raised areas 32. The sterilizer can have different constructions that accomplish raised features on which items can be placed above the water level in the bottom of the base 12. For example, a series of spaced raised integral features such as shelves or plateaus can have desired shapes, sizes and heights. The features can, but need not be, topped or covered with silicone material or another soft material that helps to grip objects that are placed on top of the features.

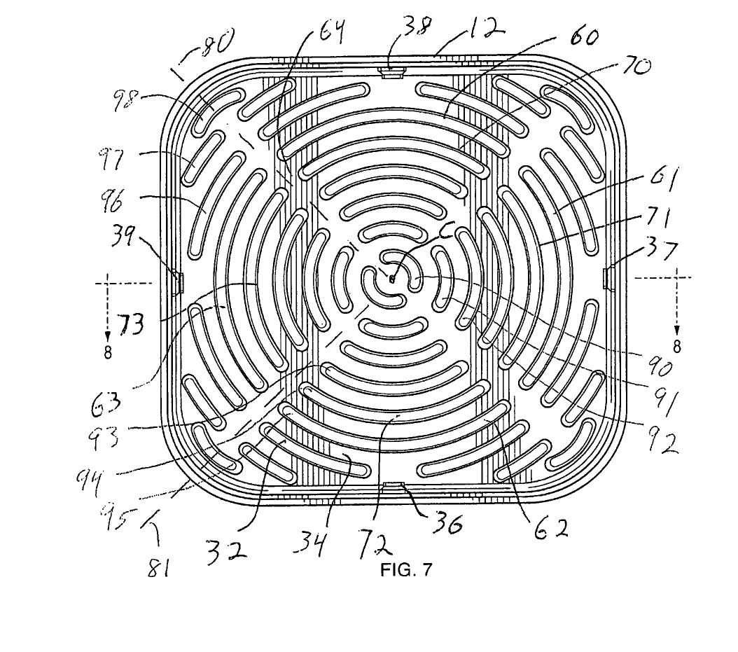

With reference to FIGS. 7 and 8, the raised areas 32 may be raised ridges such as shown in these drawings. Areas 32 may form a general pattern on the bottom wall 19 of the base 12. The raised areas 32 can be arranged generally concentrically around a central vertical axis “C” in an intermittent fashion, and in a series of spaced generally circular rings that lie along generally circular ring axes. Each segment may be arc-shape, or at least generally arc-shaped. For example, the segments need not lie exactly along circular arcs, and they do not need to be uniform in width, length or height. In this particular example, the bottom wall 19 includes a first concentric “ring” 90 having a first (smallest) radius from the central axis C, and is formed of two distinct raised areas 32. The first concentric ring 90 is closest to the central axis C. The bottom wall 19 includes a second concentric “ring” 91 having a second radius from the central axis C (greater than the first radius) and is formed of four distinct raised areas 32. The bottom wall 19 includes a third concentric “ring” 92 having a third radius from the central axis C (greater than the second radius) and is formed of four distinct raised areas 32. The bottom wall 19 includes a fourth concentric “ring” 93 having a fourth radius from the central axis C (greater than the third radius) and is formed of four distinct raised areas 32. The bottom wall 19 includes a fifth concentric “ring” 94 having a fifth radius from the central axis C (greater than the fourth radius) and is formed of four distinct raised areas 32 (the four areas are numbered 70, 71, 72, and 73). The bottom wall 19 includes a sixth concentric “ring” 95 having a sixth radius from the central axis C (greater than the fifth radius) and is formed of four distinct raised areas 32 (the four areas are numbered 60, 61, 62, and 63). The bottom wall 19 includes a seventh concentric “ring” 96 having a seventh radius from the central axis C (greater than the sixth radius) and is formed of eight distinct raised areas 32. The bottom wall 19 includes an eighth concentric “ring” 97 having an eighth radius from the central axis C (greater than the seventh radius) and is formed of eight distinct raised areas 32. The bottom wall 19 includes a ninth concentric “ring” 98 having a ninth radius from the central axis C (greater than the eighth radius) and is formed of four distinct raised areas 32, each of which lies very close to one of the four corners of the base 12.

The bottom wall 19 also defines a first axis 80 oriented perpendicularly to the central axis C and a second axis 81 oriented perpendicularly to both the first axis 80 and the central axis C. The first axis 80 and the second axis 81 generally divide the bottom wall 19 into quadrants, where all of the raised areas 32 are positioned within one of the defined quadrants except for the raised areas 32 in the ninth concentric “ring” 98. Each of the raised areas 32 in the ninth concentric “ring” 98 lie in two of the quadrants. The bottom wall 19 also includes defined spaces or gaps that run along the axes 80 and 81, which form parts of the valleys 34. The valleys 34 are also formed between raised areas 32 in adjacent concentric “rings.” For example, a valley 34 exists between the raised areas 32 that form the fourth concentric “ring” 93 and the raised areas 32 that form the fifth concentric “ring” 94.

The valleys 34 between the raised areas 32 are interconnected so that water can be poured into the base 12 at any location and still disperse evenly across the bottom wall 19. The water being evenly dispersed across the bottom wall 19 helps to ensure that the steam will evenly disperse throughout the sterilizer 10. However, interconnection of the valleys 34 is not a requirement as the steam will tend to fill interior volume or cavity 21 even if the valleys are not interconnected.

With further reference to FIGS. 7 and 8, the inner core 52 includes a plurality of vertically-oriented projections 36-39 (e.g., hooks) that extend from an inner surface 57 of the inner core 52. The projections 36-39 each define a portion 41 that is positioned inwardly of the inner surface 57 to define a gap 42 therebetween. Gap 42 forms a receptacle for receiving a relatively thin structure that can be placed into the gap 42. The portions 41 can support items such as a bottle nipple, a bottle ring, or other similar structures off of the raised areas 32 that can in part fit into a gap 42. This allows the item to hang above the surface of the boiling water. This arrangement can hold a nipple with the open side facing down toward the boiling water to help expose the inside of the nipple to steam.

The sterilizer 10 can be used to sterilize articles that fit within its interior. Up to 200 milliliters of water is placed into the base 12 in one specific, non-limiting embodiment. Items such as bottles used to feed milk or formula to infants, nipples for such bottles, bottle accessories or other baby-related devices and appliances are placed into the interior, on the bottom and/or hung from the hook structures 36-39. Other devices can include items such as pacifiers, teethers, and parts of breast pumps. The sterilizer lid 14 is positioned and the entire sterilizer is then placed into a microwave oven. Depending on the microwave wattage, the sterilizer can be heated from about two to about eight minutes. This causes the water to boil and the resulting steam will fill the interior and sterilize the parts placed into the sterilizer. The cooling time should be at least two minutes before the lid is removed. The vent 17 lets out steam so that interior pressure and temperature doesn't increase to a dangerous level.

The silicone outer skin helps to protect the user from the plastic interior which can be heated by the steam. The inner material is a thermoplastic or equivalent and should be structurally solid. The silicone parts are over-molded onto the inner core. The lid 14 is preferably but not necessarily made from the same material as the inner core.

The lid 14 can be made from two parts (the handle and the cover), which are connected together through ultrasonic welding, screws, or other fasteners or means of adhering the two parts together with the result being that the handle is integral with the cover. Or, the handle could be made in other ways, for example as an integral molded plastic part.

An alternative not shown in the drawings is an electric version with a built-in hot plate with electrically-heated coils that heat up so as to heat the water. The hot plate would be located directly bottom wall 55.

A number of implementations have been described. Nevertheless, it will be understood that additional modifications may be made without departing from the scope of the inventive concepts described herein, and, accordingly, other embodiments are within the scope of the following claims.

What is claimed is:

1. A sterilizer comprising:
a base comprising a bottom wall and a sidewall;
an elastomeric covering over at least most of the outside of the sidewall; and
a lid sized and shaped to close the base, the lid defining a vent that is adapted to vent the base.

2. The sterilizer of claim 1 wherein the lid comprises a handle.

3. The sterilizer of claim 2 wherein the vent passes through the handle.

4. The sterilizer of claim 1 wherein the bottom wall has an interior surface that defines a series of spaced raised areas.

5. The sterilizer of claim 4 wherein the raised areas are arranged generally concentrically about a central axis.

6. The sterilizer of claim 5 wherein the raised areas are generally arc-shaped.

7. The sterilizer of claim 6 wherein the raised areas comprise a plurality of generally arc-shaped segments that are spaced from one another.

8. The sterilizer of claim 7 wherein the segments are aligned along a series of generally circular axes.

9. The sterilizer of claim 8 wherein the raised areas are arranged along a plurality of radially-spaced generally circular axes.

10. The sterilizer of claim 9 wherein the raised areas are arranged such that there is a space between the segments that lie along a single generally circular axis, and wherein the spaces between segments that lie along the respective radially-spaced generally circular axes are generally radially aligned.

11. The sterilizer of claim 4 wherein the raised areas are at least substantially topped by an elastomeric material.

12. The sterilizer of claim 1 wherein the base further comprises a plurality of feet.

13. The sterilizer of claim 1 wherein the base includes a projection and wherein the elastomeric covering over at least most of the outside of the base extends over the projection of the base.

14. The sterilizer of claim 1 wherein the base has one or more thin vertical structures extending upwardly and inwardly from an interior wall of the base, wherein each thin vertical structure has a top portion that is located slightly inwardly of the interior wall so as to define an upper gap between the structure and the interior wall.

15. The sterilizer of claim 14 wherein the gap is constructed and arranged to act as a receptacle for a relatively thin structure that can be placed into the gap so as to hold the structure off of the bottom of the base.

16. A sterilizer, comprising:
a base comprising a bottom wall and a sidewall, and an elastomeric covering over at least most of the outside of the sidewall, wherein the bottom wall defines a series of raised areas that are at least substantially topped by an elastomeric material, wherein the raised areas are intermittently formed and oriented generally concentrically about a central axis; and
a lid sized and shaped to close the open top of the inner basin, the lid defining a vent that is adapted to vent the base, where the lid comprises a handle and the vent passes through the handle.

17. The sterilizer of claim 16 wherein the base includes a projection and wherein the elastomeric covering over at least most of the outside of the base extends over the projection of the base.

18. The sterilizer of claim 17 wherein the base has one or more thin vertical structures extending upwardly and inwardly from an interior wall of the base, wherein each thin vertical structure has a top portion that is located slightly inwardly of the interior wall so as to define an upper gap between the structure and the interior wall.

19. The sterilizer of claim 18 wherein the gap is constructed and arranged to act as a receptacle for a relatively thin structure that can be placed into the gap so as to hold the structure off of the bottom of the base.
